(19) United States
(12) Patent Application Publication
(10) Pub. No.: US 2002/0022810 A1
(43) Pub. Date: Feb. 21, 2002

(54) Non-linear flow restrictor for a medical aspiration system

(76) Inventor: Alex Urich, Mission Viejo, CA (US)

Correspondence Address: IRELL & MANELLA LLP, 840 NEWPORT CENTER DRIVE, SUITE 400, NEWPORT BEACH, CA, 92660, US

(21) Appl. No.: 09/973,280

(22) Filed: Oct. 8, 2001

Related U.S. Application Data
(60) Provisional application No. 60/169,422, filed on Dec. 7, 1999.

(51) Int. Cl. A61B 17/32; A61M 1/00; A61B 10/00

(52) U.S. Cl. 604/317; 606/167; 606/170; 600/565

(57) ABSTRACT

A non-linear flow restrictor that limits the maximum flowrate in a medical aspiration system. The flow restrictor changes the direction of fluid flow to generate non-linear effects in the fluid. This creates a non-linear relationship between the pressure within the system and the flowrate of the fluid. The non-linear relationship may define a pressure versus flowrate curve that has a flat portion where the flowrate does not increase with an increase in pressure.

### CROSS-REFERENCE TO RELATED APPLICATION

The present application is a continuation-in-part of Application No. 546,804, filed on Apr. 11, 2000, pending, which claims the benefit of U.S. Provisional Application No. 60/169,422, filed Dec. 7, 1999.

### BACKGROUND OF THE INVENTION

1. Field of the Invention

The present application relates to a medical aspiration system.

2. Background Information

The lens of a human eye may develop a cataracteous condition which affects a patients vision. Cataracteous lenses are sometimes removed and replaced in a procedure commonly referred to as phacoemulsification. Phaco procedures are typically performed with an ultrasonically driven handpiece which is used to break the lens. The broken lens is removed through an aspiration line that is coupled to the handpiece.

The handpiece has a tip which is inserted through an incision in the cornea. The handpiece typically contains a number of ultrasonic transducers that convert electrical power into a mechanical oscillating movement of the tip. The distal end of the tip has an opening that is in fluid communication with the aspiration line. The distal end of the tip also has a sleeve which has an opening in fluid communication with an irrigation line. The irrigation line is typically connected to a bottle that can provide irrigation fluid to the surgical site.

The oscillating movement of the tip will break the lens into small pieces. The lens pieces and irrigation fluid are drawn into the aspiration line through the opening of the tip. When performing a phaco procedure it is essential to maintain a positive pressure within the anterior chamber of the eye. A negative pressure may cause the cornea to collapse. To maintain a positive chamber pressure the system is configured to provide a flowrate through the irrigation tube that is greater than the flowrate through the aspiration tube.

It has been found that the aspiration tube may become occluded during a procedure. The occlusion will increase the vacuum pressure within the aspiration line. When the occlusion is cleared the anterior chamber may be instantaneous exposed to a high vacuum pressure. The vacuum pressure may cause the cornea to collapse. It would be desirable to provide an aspiration system that minimizes the effects of a cleared occlusion within an aspiration tube of the system.

### BRIEF SUMMARY OF THE INVENTION

A non-linear flow restrictor that limits the flowrate of a fluid flowing through a medical aspiration system.

### BRIEF DESCRIPTION OF THE DRAWINGS

FIG. 1 is an illustration of a medical system which contains an aspiration tube that has a relatively high fluidic resistance;

FIG. 2 is an illustration of a non-linear flow restrictor;

FIG. 3 is a graph showing a non-linear relationship between a vacuum pressure and a flowrate of a fluid flowing through the flow restrictor.

### DETAILED DESCRIPTION

Disclosed is a non-linear flow restrictor that limits the maximum flowrate in a medical aspiration system. The flow restrictor changes the direction of fluid flow to generate non-linear effects in the fluid. This creates a non-linear relationship between the pressure within the system and the flowrate of the fluid. The non-linear relationship may define a pressure versus flowrate curve that has a flat portion where the flowrate does not increase with an increase in pressure.

When used in an aspiration system to perform an opthtalmic procedure, the non-linear flow restrictor will limit a rapid raise in flowrate due to an occlusion in the system and prevent corneal collapse. Although use of an aspiration system used to perform opthtalmic procedures is disclosed and described, it is to be understood that the non-linear flow restrictor can be used in an aspiration system used to perform other medical procedures.

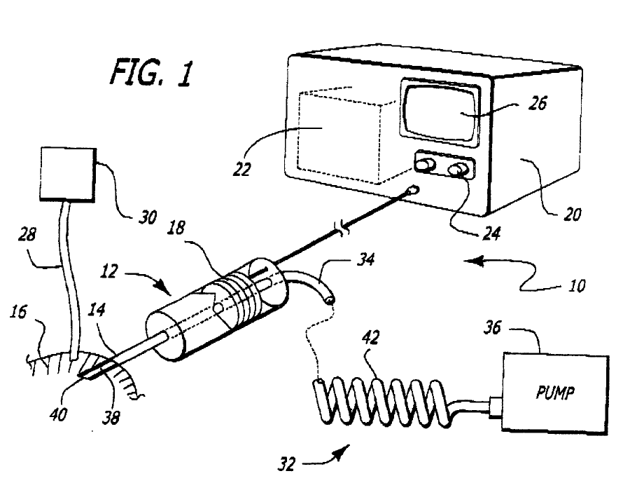

Referring to the drawings more particularly by reference numbers, FIG. 1 shows an embodiment of a medical system 10 of the present invention. The system 10 may include an ultrasonically driven handpiece which has a tip 14 that can be inserted into a cornea 16. The tip 14 may also be referred to as a cutting element. The handpiece 12 may include one or more ultrasonic transducers 18 that convert electrical power into mechanical movement of the tip 14. The handpiece 12 is typically held by a surgeon who performs a surgical procedure with the system 10. By way of example, the system 10 can be used to perform a phacoemulsification procedure to break and aspirate a lens of the cornea 16.

The handpiece 12 may be connected to a console 20 of the system 10. The console 20 may contain a control circuit 22 that provides a driving signal to the transducers 18. The console 20 may have input knobs or buttons 24 that allow the surgeon to vary different parameters of the system 10. The console 20 may also have a readout display 26 that provides an indication of the power level, etc. of the system 10.

The system 10 may include an irrigation tube 28 that is connected to an irrigation bottle 30. The irrigation tube 28 can be inserted into the cornea 16. The irrigation bottle 30 may contain an irrigation fluid that flows into the cornea 16 through the irrigation tube 28.

The medical system 10 may further have an aspiration system 32 that aspirates the irrigation fluid and broken lens out of the cornea 16. The aspiration system 32 may include an aspiration tube 34 that is connected to the handpiece 12 and a vacuum pump 36. By way of example, the vacuum pump 36 may be a peristaltic pump or a Venturi type device. The aspiration tube 34 is in fluid communication with an inner channel 38 and an opening 40 of the tip 14. The vacuum pump 36 creates a negative pressure within the aspiration tube 34 to induce a flow of irrigation fluid and emulsified tissue out of the cornea 16. The pump 36 is configured so that the flowrate through the irrigation tube 28 is slightly greater than the flowrate through the aspiration tube 34.

The aspiration tube 34 has a relatively large fluidic resistance to create a large fluid inertia in the aspiration system 32. The large inertia minimizes instantaneous changes in the flowrate of irrigation fluid through the aspiration tube 34. Thus if an occlusion is cleared within the aspiration tube 34 the large fluidic resistance will restrict the variation in aspiration fluid flow and minimize the probability of a cornea collapse event.

It has been found that having an aspiration tube 34 at least 8 feet long will provide a fluidic resistance sufficient to minimize the effects of an occlusion during a phaco procedure. A tube 34 less than 8 feet may not provide enough fluidic resistance to minimize changes in flowrate through the aspiration tube 34. The aspiration tube 34 may contain a plurality of pre-formed coils 42 to shorten the effective length of the tube 34. Coiling the aspiration tube 34 also increases the fluidic resistance of the tube 34.

In one embodiment the aspiration tube 34 may have a pre-coiled straight length of 12 feet. There may be 50 pre-formed coils 42, each having a diameter of 0.5 inches. The inner diameter of the tube 34 may be 0.065 inches. It has been found that such an embodiment will reduce the flowrate generated by a vacuum pressure of 600 millimeters of mercury (mmHg) approximately 10 times from a straight uncoiled tube of equal length. The coils 42 repeatedly change the direction of fluid flow and create a non-linear relationship between the pressure and the flowrate within the tube. The coils 42 create a non-linear flow restrictor.

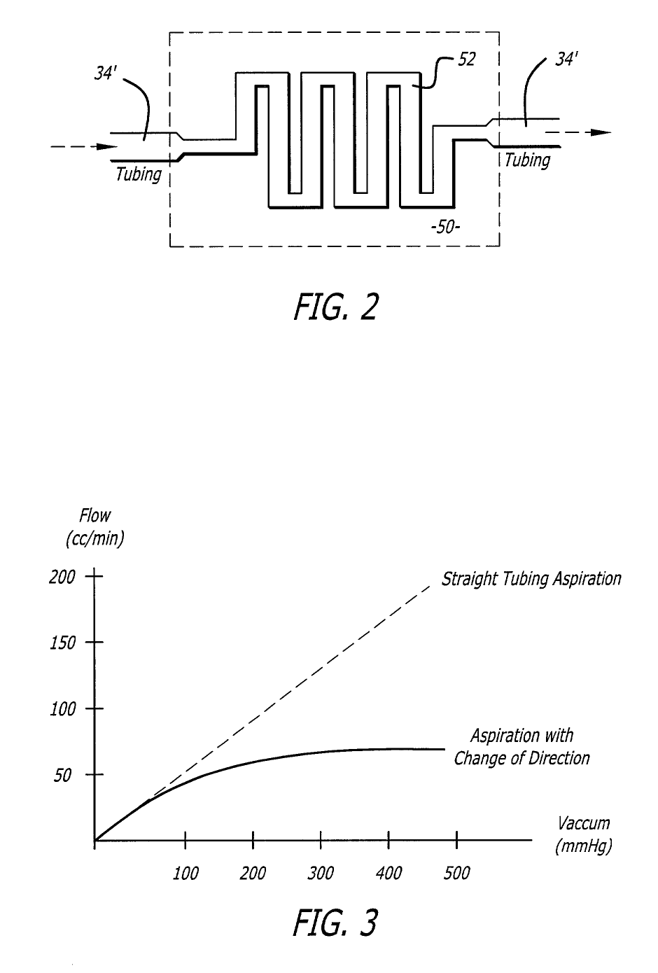

FIG. 2 shows another embodiment of a non-linear flow restrictor 50. The flow restrictor 50 may include a plurality of bends 52 in an aspiration tube 34′. The bends 52 change the direction of fluid flow and create a nonlinear relationship between the flowrate and pressure in the tube 34′. The flow restrictor 50 shown in FIG. 2 may be substituted for the coils 42 showing in FIG. 1. Alternatively, the restrictor 50 may be included with the coils 42.

FIG. 3 shows a graph of pressure versus flowrate for the flow restrictor 50 with 50 bends and an inner diameter of 0.065 inches. The restrictor 50 was coupled to a Venturi pump. As shown by the dotted line, a straight tube will generate a linear relationship between variations in the vacuum pressure and the flowrate of fluid through the aspiration tube. The flow restrictor of the present invention creates a non-linear relationship between variations in the vacuum pressure and the flowrate as shown by FIG. 3. The curve established by the restrictor has a flat non-linear portion such that an increase in vacuum pressure will not increase the flowrate of the fluid. This prevents excessive fluid flow through the aspiration system, a characteristic that is particularly useful when used in an opthtalmic procedure. The curve including the location of the flat portion, may be varied by changing the number of bends and/or the inner diameter of the flow restrictor. The coiled tube 42 shown in FIG. 1 may also create a curve having the characteristics depicted in FIG. 3.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

Although the pre-formed coils 42 are shown in a cylindrical “telephone cord” arrangement, it is to be understood that the coils 42 may be provided in a different configuration. For example, the coils 42 may be nested or overlapping.

Additionally, although coiled and bent tubes have been shown as examples of a non-linear flow restrictor, it is to be understood that other embodiments which change the direction of fluid flow and create non-linear fluid effects are within the scope of this invention.

What is claimed is:

1. A medical aspiration system, comprising:
a pump; and,
a non-linear flow restrictor coupled to said pump.

2. The system of claim 1, wherein said non-linear flow restrictor changes a direction of fluid flow.

3. The system of claim 1, wherein said pump is a peristaltic device.

4. The system of claim 1, wherein said pump is a venturi device.

5. The system of claim 1, wherein said non-linear flow restrictor includes a tube that has a plurality of bends.

6. A medical cutting system, comprising:
a handpiece;
a cutting element attached to said handpiece;
a pump coupled to said handpiece; and,
a non-linear flow restrictor coupled to said pump and said handpiece.

7. The system of claim 6, wherein said non-linear flow restrictor changes a direction of fluid flow.

8. The system of claim 6, wherein said pump is a peristaltic device.

9. The system of claim 6, wherein said pump is a venturi device.

10. The system of claim 6, wherein said non-linear flow restrictor includes a tube that has a plurality of bends.

11. A medical aspiration system, comprising:
pump means for creating a flow of fluid that has a pressure and a flowrate; and,
flow restrictor means for creating a non-linear relationship between variations in the pressure and the flowrate of the fluid.

12. The system of claim 11, wherein said flow restrictor means changes a direction of fluid flow.

13. The system of claim 11, wherein said pump means includes a peristaltic device.

14. The system of claim 11, wherein said pump means includes a venturi device.

15. The system of claim 11, wherein said flow restrictor means includes a tube that has a plurality of bends.

16. A medical cutting system, comprising:
a handpiece;
a cutting element attached to said handpiece;
pump means for creating a flow of fluid that has a pressure and a flowrate; and,
flow restrictor means for creating a non-linear relationship between variations in the pressure and the flowrate of the fluid.

17. The system of claim 16, wherein said non-linear flow restrictor means changes a direction of fluid flow.

18. The system of claim 16, wherein said pump is a peristaltic device.

19. The system of claim 16, wherein said pump is a venturi device.

20. The system of claim 16, wherein said non-linear flow restrictor includes a tube that has a plurality of bends.

21. A method for aspirating fluid in a medical system, comprising:
creating a flow of fluid that has a pressure and a flowrate; and,
restricting the flow of fluid so that a variation in the pressure will create a non-linear change in the flowrate.

22. The method of claim 21, changing a direction of the fluid flow.
